(19) United States
(12) Patent Application Publication
(10) Pub. No.: US 2015/0126526 A1
(43) Pub. Date: May 7, 2015

(54) CO-CRYSTAL OF AN ANTIDEPRESSANT COMPOUND

(71) Applicant: Dipharma Francis S.r.l, Baranzate (MI) (IT)

(72) Inventors: Nicolas TESSON, Barcelona (ES); Jordi de Mier VINUÉ, Barcelona (ES)

(73) Assignee: DIPHARMA FRANCIS S.R.L., Baranzate (MI) (IT)

(21) Appl. No.: 14/532,162

(22) Filed: Nov. 4, 2014

(30) Foreign Application Priority Data
Nov. 6, 2013 (IT) MI2013A001839

(51) Int. Cl. A61K 47/12; C07D 405/12

(52) U.S. Cl. CPC A61K 47/12; C07D 405/12
USPC 514/254.09; 544/373

(57) ABSTRACT

Co-crystal of a serotonin reuptake inhibitor, in stable form.

### TECHNICAL FIELD

The present invention relates to a new co-crystal of Vilazodone hydrochloride having better properties than Vilazodone hydrochloride itself, a process for its preparation, its use in therapy and a pharmaceutical composition containing it.

### BACKGROUND ART

Vilazodone is the name of the chemical compound 5-(4-[4-(5-cyano-1H-indol-3-yl)butyl]piperazin-1-yl)benzofuran-2-carboxamide of formula (I). This compound is a serotonin reuptake inhibitor and acts as a 5-HT1A partial receptor agonist.

Vilazodone is marketed as hydrochloride salt and is used in adults humans in the treatment of severe depressing conditions at dosages of 10, 20 and 40 mg.

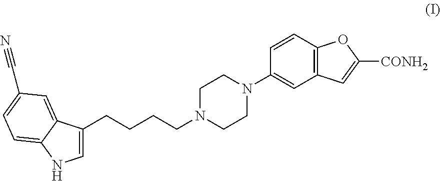 [SMILES: N#Cc1ccc2[nH]cc(CCCCN3CCN(c4ccc5oc(C(N)=O)cc5c4)CC3)c2c1]

Vilazodone is known from U.S. Pat. No. 5,532,241, which also discloses the synthesis thereof. EP 1 397 357 discloses various crystalline forms of vilazodone hydrochloride and dihydrochloride, both anhydrous and hydrates ones, and also amorphous Vilazodone hydrochloride.

As known, the co-crystals, are crystals that contain two or more molecules that are not the same. The two or more molecules that form the co-crystal do not have ionic or covalent type bonds, but they have weaker interactions, such as hydrogen bonds. Through the co-crystallization of a first compound with a second different compound, defined as “co-former”, a new solid form can form, having better properties than a first compound. For example, a co-crystal can have different dissolution and solubility properties and therefore different bioavailability, compared with the same compound or a salt thereof.

In the pharmaceutical field the first compound is often known as Active Pharmaceutical Ingredient (API). The other different compound of the co-crystal, the “co-former”, is a pharmaceutically acceptable compound. Co-crystals of and API can be advantageously used in therapy in an alternative method of administration of the same API.

In fact, a pharmaceutical composition containing a co-crystal of an API can have more advantageous properties compared with the one that contains the only API as active ingredient.

The possibility of formation of a co-crystal of an API with a co-former cannot be foreseen, and, especially, its formation is not always possible. Furthermore, no way it is possible to foresee the physical, chemical and biologic properties of the co-crystal of an API, till this is not actually obtained.

### SUMMARY OF THE INVENTION

It has now been found a co-crystal of Vilazodone hydrochloride (API) L-tartaric acid (co-former), in particular a co-crystal of Vilazodone hydrochloride L-tartaric acid, wherein the molar ratio between API and coformer is 2:1.

The invention also relates to a process for the preparation of said co-crystal, its use in a therapeutic method of treatment and pharmaceutical composition containing it, as active ingredient, and at least an excipient and/or pharmaceutically acceptable carrier.

### DESCRIPTION OF THE FIGURES AND ANALYTICAL METHODS

The co-crystal has been characterized through X ray diffraction from crystalline powders (XRPD) (X-ray powder diffraction), by nuclear magnetic resonance spectrometer ¹H-NMR, CP-MAS ¹⁵N-NMR, by differential scanning calorimetry (DSC) and by thermo-gravimetric analysis (TGA).

The X-ray powder diffraction (XRPD) spectrum was collected with a PAnalytical X'Pert diffractometer for powders under the following operating conditions: Radiation CuKα (λ=1.5418 Å), scanning: 2θ angle range of 3° to 40°, for a rate of 17.6°/min.

The ¹H-NMR spectrum was collected with Varian Mercury 400 spectrometer, using DMSO-d6, as solvent.

The CP-MAS ¹⁵N-NMR spectra were collected with a Bruker 400 MHz spectrometer with a probe of 4 mm and a rotation rate of 10 KHz. The time range for measuring was of 22 h. Two different experiments were carried out using a different contact time, 2500 and 120 μs.

The DSC thermogram was acquired by a Mettler-Toledo DSC 822e differential scanning calorimeter under the following operating conditions: aluminum capsules, range 30-300° C., at the rate of 10° C./min, with nitrogen as purge gas (50 ml/min).

The TGA thermogram was acquired through a Mettler-Toledo TGA/SDTA 851e thermogravimetric analyzer, in the following operating conditions: alumina capsules, range 30-300° C. with a rate of 10° C./min, with nitrogen as purge gas (50 ml/min).

The particle size and the D₅₀ was determined with the known laser light scattering technique using a Malvern Mastersizer 3000 instrumentation.

FIG. 1: XRPD spectrum of the Vilazodone hydrochloride L-tartaric acid co-crystal.

FIG. 2: ¹H-NMR spectrum of the co-crystal of Vilazodone hydrochloride with L-tartaric acid.

FIG. 3: DSC thermogram of the co-crystal of Vilazodone hydrochloride with L-tartaric acid.

FIG. 4: TGA thermogram of the co-crystal of Vilazodone hydrochloride with L-tartaric acid.

FIG. 5: ¹⁵N-NMR spectrum of the co-crystal of Vilazodone hydrochloride with L-tartaric acid—contact time of 2500 μs.

FIG. 6: ¹⁵N-NMR spectrum of the co-crystal of Vilazodone hydrochloride with L-tartaric acid—contact time of 120 μs.

### DETAILED DESCRIPTION OF THE INVENTION

The present invention relates to a co-crystal of vilazodone hydrochloride (API) L-tartaric acid (co-former), in particular a co-crystal of vilazodone hydrochloride L-tartaric acid having molar ratio 2:1 between the API and the co-former.

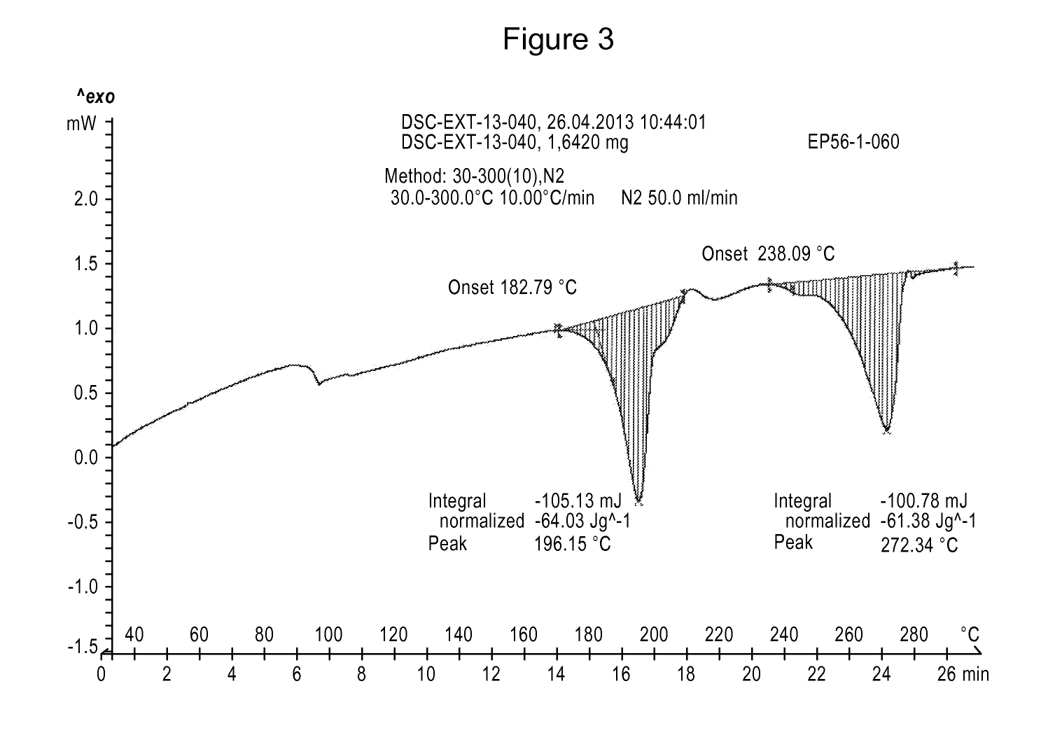

The co-crystal has a DSC thermogram as substantially shown in FIG. 3, having a first peak at about 180-190° C. and a second peak at about 240-280° C. In particular, the first peak at about 180-190° C. represents the fusion of the co-crystal, followed by the degradation of the tartaric acid and the recrystallization of vilazodone hydrochloride, while the second peak at about 240-280° C. represents the fusion of vilazodone hydrochloride.

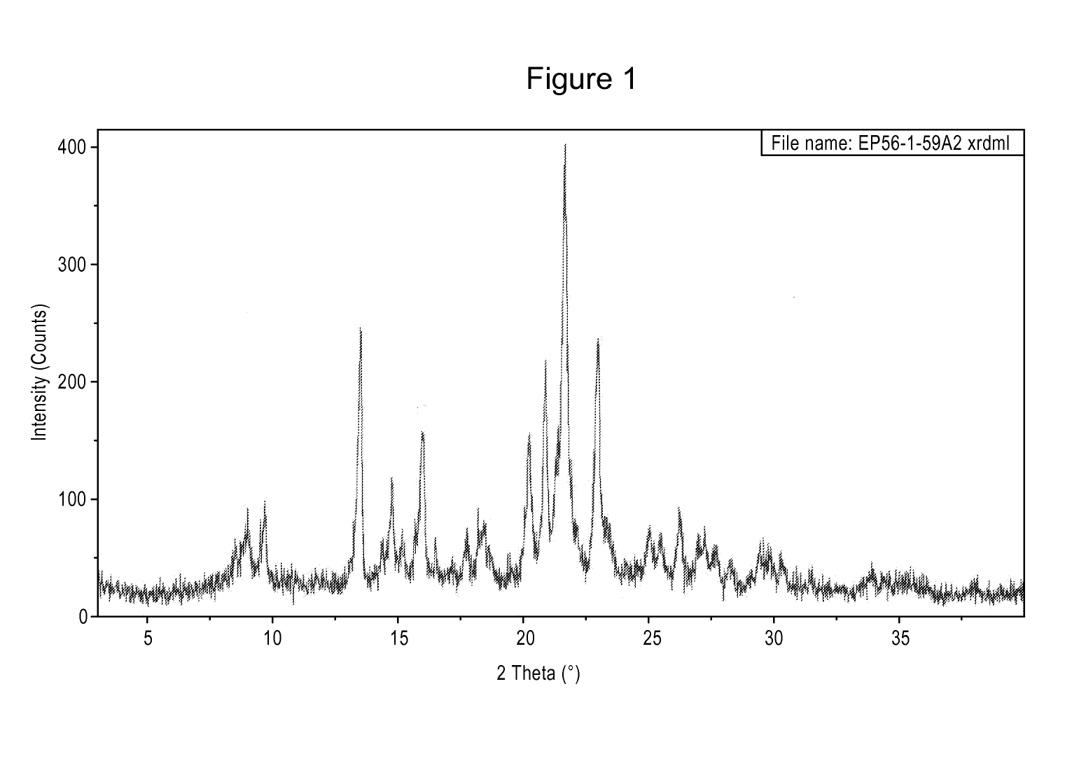

The same co-crystal has a XRPD spectrum as substantially shown in FIG. 1, wherein the most intense peaks fall at 9.0; 9.7; 13.5; 14.8; 16.0; 16.5; 20.2; 20.9; 21.6; 23.0; 25.1 and 26.3±0.2° in 2θ.

The cell data of the co-crystal, obtained starting from the position of the diffraction peaks using the program Topas by Bruker AXS version 4.2, are the following:

Spatial group: C2/c; a: 23.9623 Å; b: 11.5162 Å; c: 20.9511 Å; and β: 71.63°.

This cell data, allowing the indexation of all the observed peaks in the diffractometer, show that the sample is made of a sole crystalline phase and not of a mixture of phases, therefore vilazodone hydrochloride and L-tartaric acid are part of a sole crystalline phase.

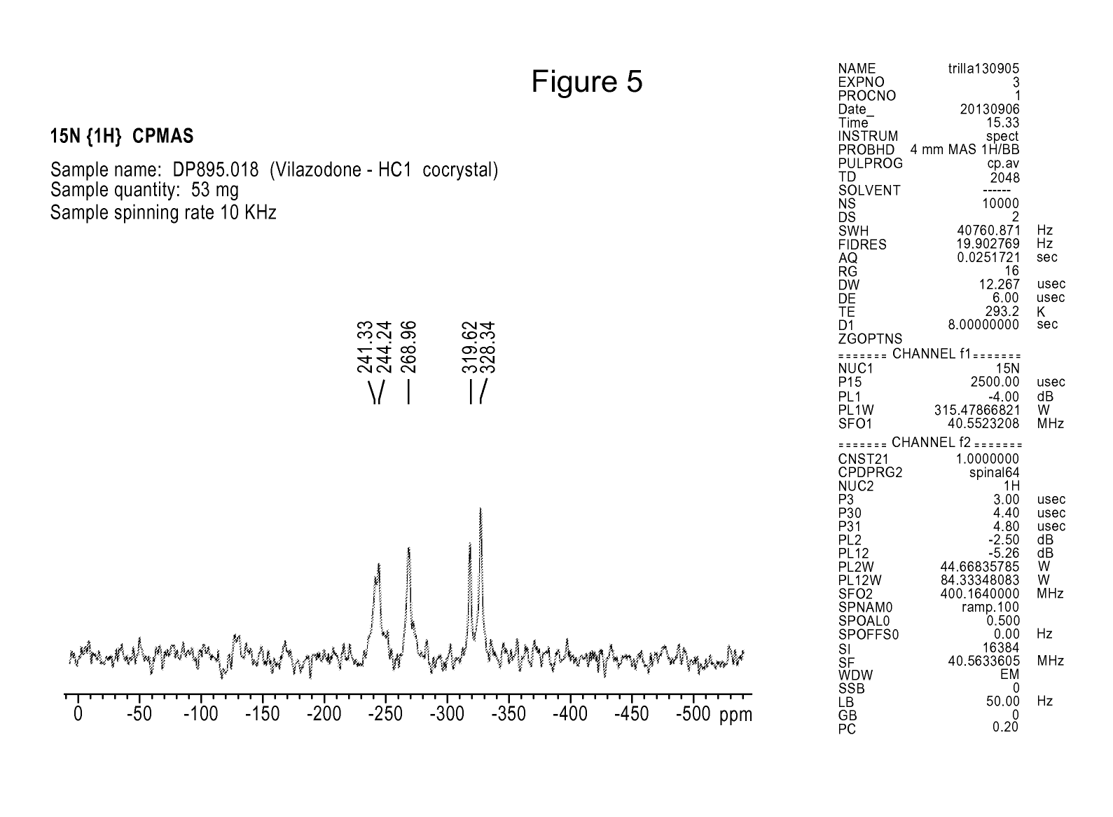

The same co-crystal has a CP-MAS ¹⁵N-NMR spectrum registered with a contact time of 2500 μs, as shown in FIG. 5, wherein four main peaks are observed at about −243; −269; −320; and −328 ppm, related to the nitromethane signal, using glycine as external standard (−347.54 ppm).

The fifth possible signal, pertaining to the nitrogen of nitrile group, is not present because it cannot be always observed in the mentioned working conditions, as known from Bioresource Technology, 2007, 98, 1494-1500.

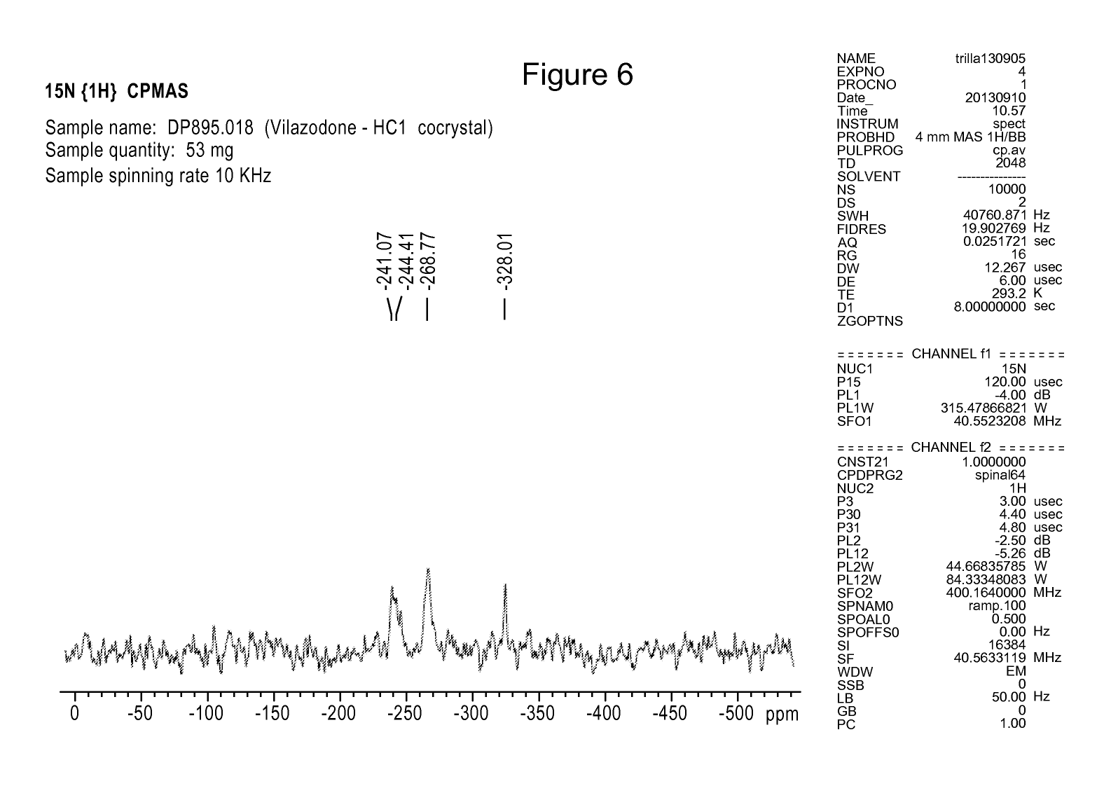

Furthermore, the co-crystal presents a CP-MAS ¹⁵N-NMR spectrum registered with a contact time of 120 μs, as shown in FIG. 6, wherein only three main peaks are observed at about −243; −269; and −328 ppm. As known in the art, low contact times render visible only the signals pertaining to the nitrogen atom directly bound to the hydrogen atoms. The absence of the signal at −320 ppm therefore demonstrate the presence of a non-protonated nitrogen in the molecule of vilazodone. Therefore, it can be safely stated tartaric acid does not form a salt with Vilazodone, but a co-crystal, since no proton-transfer occurs.

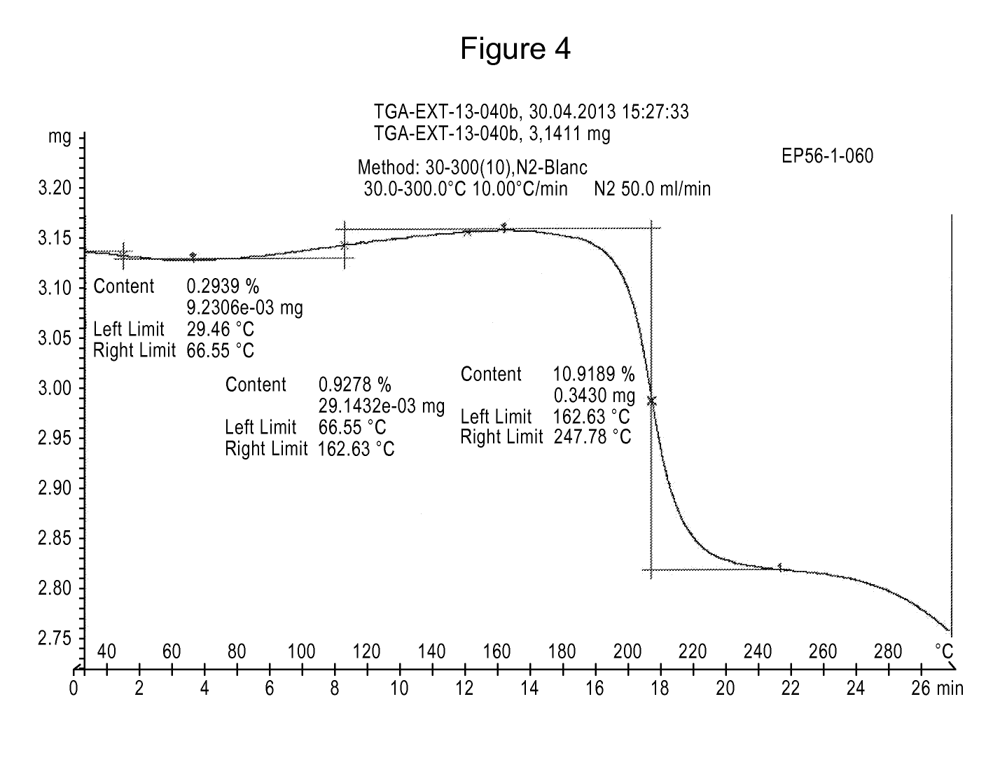

The same co-crystal is a substantially anhydrous product, as shown by the TGA thermogram at FIG. 4, showing the absence of water or of other crystallization solvents.

A second object of the present invention is a process for the preparation of a co-crystal of vilazodone hydrochloride (API) L-tartaric acid (co-former), as defined above, comprising:
a) preparing a suspension of vilazodone hydrochloride in a keto solvent;
b) adding L-tartaric acid to the suspension; and
c) recovering the solid.

Vilazodone hydrochloride used as starting material for the preparation of the suspension can be any form of vilazodone hydrochloride known in the art, which is scarcely soluble or insoluble in a keto solvent, for example vilazodone hydrochloride Form VI, which is substantially hemihydrate, known by EP 1 397 357.

The keto solvent can be a straight or branched C₃-C₆ ketone, for example acetone, methylethylketone, 3-pentanone or methylisobutylketone, preferably methylisobutylketone (MIBK); or a mixture thereof with water, in the latter case, preferably saturated with water.

The amount of keto solvent to the amount of vilazodone hydrochloride can be comprised between about 15 g/ml and 25 g/ml, more preferably about 20 g/ml.

The addition of L-tartaric acid to the suspension is preferably carried out by adding solid L-tartaric acid.

The weight/weight ratio between vilazodone hydrochloride and tartaric acid can be comprised for example between about 0.8 and about 2.5, preferably between about 1 and about 2, more preferably between 1.5 and 1.7.

The suspension of Vilazodone hydrochloride before and after the addition of L-tartaric acid, is typically maintained at room temperature.

In order to promote the formation of the co-crystal, the so obtained mixture at step b) can be maintained under stirring for a range of time comprised between about 6 and 16 hours, typically between 8 and 12 hours.

Besides, in order to promote the formation of the co-crystal, the precipitation can be triggered by seeding the suspension obtained at step b) a with a co-crystal, previously obtained according to the above disclosed process.

The recovery of the white solid of the co-crystal can be carried out by known techniques to the man skilled in the art, for example by filtration through a Bückner filter or by centrifugation.

The solid is then preferably washed with a keto solvent as defined above, preferably the same solvent used at step a), and then dried, preferably at reduced pressure, according to known methods.

The co-crystal of vilazodone hydrochloride L-tartaric acid has been surprisingly found to be a stable solid both from a chemical and physical point of view, as proven for example by the results of the tests disclosed here below.

The co-crystal of vilazodone hydrochloride L-tartaric acid, closed in a sealed vial, after maintenance for one month at room temperature, nevertheless shows a XRPD spectrum as substantially shown in FIG. 1.

The co-crystal of Vilazodone hydrochloride L-tartaric acid, heated, under vacuum, at 45° C. for two days, nevertheless shows a XRPD spectrum as substantially shown in FIG. 1. The same co-crystal, heated, under vacuum, at 75° C. for two days, or at 90° C. for one day, shows a XRPD spectrum as substantially shown in FIG. 1.

The co-crystal of vilazodone hydrochloride L-tartaric acid, maintained for three days at a relative humidity (RH) of about 71-76%, in particular of 75%, at room temperature, typically around 19-23° C., anyway shows a XRPD spectrum as substantially shown at FIG. 1.

These results are the proof that the new polymorph is stable both to the thermal stress and to the presence of moisture, and besides it does not decompose to form a polymorph of vilazodone hydrochloride.

The so obtained co-crystal has D₅₀ value comprised between about 25 and 250 μm. If desired, this value can be reduced by micronization or fine grinding.

A further object of the present invention is a pharmaceutical composition comprising the co-crystal of vilazodone hydrochloride L-tartaric acid, as defined above, as active ingredient and at least a pharmaceutically acceptable excipient and/or carrier.

Such pharmaceutical composition can be prepared in a pharmaceutical dosage form according to the known techniques. The dosage of the active ingredient present in such composition can be the one commercially used in therapy for vilazodone hydrochloride.

A further object of the invention is a co-crystal of Vilazodone hydrochloride L-tartaric acid, as defined above, for use as a medicament, in particular as serotonin reuptake inhibitor.

A further object of the invention is the use of a vilazodone hydrochloride L-tartaric acid co-crystal, as defined above, for the preparation of a medicament useful as serotonin reuptake inhibitor.

Therefore, a further object of the present invention is a method for treatment of a human being, in need of a serotonin reuptake inhibitor, for example in a method for preventing and treating depressing conditions, bipolar disorders, anxious conditions, maniacal conditions, dementia, sexual dysfunctions, sleeping disorders, psychiatric disorders, cerebral stroke, food disorders, obesity, fibromyalgia, comprising administration to said human being of a therapeutically effective amount of the co-crystal of vilazodone hydrochloride L-tartaric acid co-crystal, as defined above.

The following example illustrate the invention.

### Example 1

### Preparation of Vilazodone Cloridrato (API) Acid L-Tartaric Acid (Co-Former) Co-Crystal

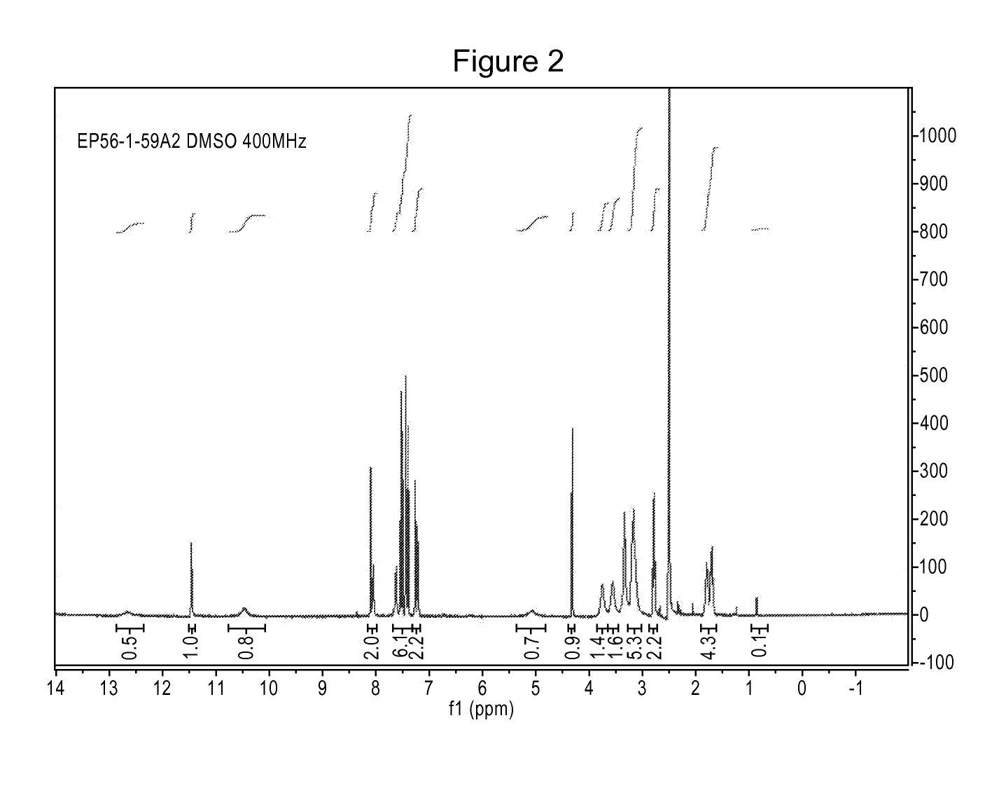

In a 25 ml flask with mechanical stirrer, containing vilazodone hydrochloride Form VI (242 mg; 0.50 mmols) in suspension with 5 ml of MIBK, L-tartaric acid is added (48 mg; 0.32 mmols; 0.65 eq.). The resulting mixture is stirred overnight at room temperature. A white solid forms, it is filtered on a Büchner filter and washed with MIBK (3×1 mL) After drying under vacuum at 75° C. for two days, 197 mg of product are obtained (yield: 63%). The solid has a (XRPD) spectrum as shown at FIG. 1, wherein the most intense diffraction peaks fall at 9.0; 9.7; 13.5; 14.8; 16.0; 16.5; 20.2; 20.9; 21.6; 23.0; 25.1 and 26.3±0.2° in 2θ; the ¹H-NMR spectrum shown at FIG. 2; the DSC thermogram shown in FIG. 3; the TGA thermogram shown in FIG. 4; and the CP-MAS ¹⁵N-NMR spectra shown in FIGS. 5 and 6.

1. Co-crystal of vilazodone hydrochloride (API) L-tartaric acid (co-former).

2. A co-crystal according to claim 1, having a molar ratio 2:1 between the API and the co-former.

3. A co-crystal according to claim 1, in substantially anhydrous form.

4. A co-crystal according to claim 1, having a XRPD, as substantially shown in FIG. 1, wherein the most intense diffraction peaks fall at 9.0; 9.7; 13.5; 14.8; 16.0; 16.5; 20.2; 20.9; 21.6; 23.0; 25.1; and 26.3±0.2° in 2q (CuKa).

5. A co-crystal according to claim 1, having a DSC as substantially shown in FIG. 3, having a first peak at about 180-190° C. and a second peak at about 240-280° C.

6. Process for the preparation of a co-crystal of vilazodone hydrochloride (API) L-tartaric acid (co-former), as defined in claim 1, comprising:
a) preparing a suspension of vilazodone hydrochloride in a keto solvent;
b) adding L-tartaric acid to the suspension; and
c) recovering the solid.

7. Process according to claim 4, wherein the suspension obtained at step b) is seeded with a co-crystal of vilazodone hydrochloride (API) L-tartaric acid (co-former).

8. A process according to claim 4, wherein vilazodone hydrochloride used as starting material is a vilazodone hydrochloride, which is scarcely soluble or insoluble in a keto solvent.

9. A process according to claim 6, wherein the keto solvent is a C₃-C₆ ketone, or a mixture thereof with water.

10. Process according to claim 6, wherein the keto solvent is methylisobutylketone.

11. Pharmaceutical composition comprising a co-crystal of vilazodone hydrochloride L-tartaric acid, as defined in claim 1, as active ingredient, and at least a pharmaceutically acceptable excipient and/or carrier.

12. A co-crystal of vilazodone hydrochloride L-tartaric acid, as defined in claim 1, for use as a medicament.

13. Method of treatment of a human being in need of a serotonin reuptake inhibitor, comprising administering to the human being a therapeutically effective amount of the vilazodone hydrochloride L-tartaric acid co-crystal, as defined in claim 1.
